(19) United States
(12) Patent Application Publication
(10) Pub. No.: US 2015/0252396 A1
(43) Pub. Date: Sep. 10, 2015

(54) METHOD FOR INCREASED PHOSPHORUS RECOVERY FROM ORGANIC RESIDUES

(71) Applicant: FRAUNHOFER-GESELLSCHAFT ZUR FÖRDERUNG DER ANGEWANDTEN FORSCHUNG E.V., München (DE)

(72) Inventors: Alejandra Campos, Stuttgart (DE); Jennifer Bilbao, Stuttgart (DE); Susanne Zibek, Reutlingen (DE); Maria Soledad Stoll, Marktheidenfeld (DE); Siegfried Egner, Adelsheim (DE); Thomas Hirth, Buhl (DE)

(73) Assignee: Fraunhofer-Gesellschaft zur Förderung der angewandten Forschung e.V., München (DE)

(21) Appl. No.: 14/437,737

(22) PCT Filed: Oct. 31, 2013
(86) PCT No.: PCT/EP2013/072810

§ 371 Date: Apr. 22, 2015

(30) Foreign Application Priority Data
Nov. 14, 2012 (DE) 10 2012 220 810.0

(51) Int. Cl. C12P 9/00; C12P 3/00

(52) U.S. Cl. CPC C12P 9/00; C12P 3/00

(57) ABSTRACT

The invention relates to a method for recovering organic and inorganic phosphorous compounds from solid components of organic residues. The method consists of the following steps: the organic residues are separated in a first solid phase and a first liquid phase; the first solid phase is mixed with the treatment water to form a solution; bivalent ions contained in the solution are converted into difficult to dissolve or complexed chemical compounds: organic phosphorous compounds are enzymatically reacted to form inorganic phosphates; the solution is separated in a second solid phase and a second liquid phase; the inorganic phosphate is recovered or separated from the second liquid phase; ammonium salts are recovered or separated from the second liquid phase; bivalent ions obtained in the first liquid phase are separated in the form of salts; the second solid phase is dried; the second phase is mixed and pelletized with phosphate salts, ammonium salts and salts of the bivalent ions which are obtained in the previous deposition processes.

The invention concerns a method for recovery of organic and inorganic phosphorus compounds from solid components of organic residues.

Organic residues, comprising animal manure, fermenting residues from anaerobic fermentation or other residues of organic origin, contain a high degree of phosphate, nitrogen and potassium and are therefore used as fertilizer in farming applications. This produces economic and ecological benefits, since some of the mineral fertilizer can be spared.

It is known how to separate the organic residues before being applied to the field into a liquid phase and a solid phase by means of a mechanical process, such as centrifuging. A large portion, around 70%, of the phosphorus remains in the solid phase. On the other hand, around 70-80% of the nitrogen and potassium contained in the residues are in the liquid phase.

Consequently, a substantial surplus of phosphorus gets into the soil when the solid phase is applied, the amount of which is determined by the nitrogen requirement. The oversaturation of the soil with phosphorus has negative impact on the environment. Thus, for example, the phosphorus is washed away with the rain and thus gets into bodies of water, resulting in eutrophication, i.e., increased plant growth, such as algae growth, due the a surplus of nutrients.

It is conceivable to transport the liquid phase to districts having a deficit of nutrients. However, the transport of large amounts of liquid is very energy and cost intensive.

As an alternative solution, it is known how to precipitate the phosphorus contained in the liquid phase in the form of hard to dissolve phosphate salts by crystallization.

Due to the low phosphorus content of the liquid phase, less than 30%, the yield of phosphate salts like magnesium ammonium phosphate, also known as struvite, or calcium phosphate, is very low and therefore uneconomical.

The high phosphorus content is attributable to the presence of organic phosphorus compounds and insoluble inorganic phosphates, especially potassium phosphates and magnesium phosphates, in the solid phase. However, only the phosphorus inorganically bound as PO₄³⁻ is available to the plants.

The phosphorus from organic phosphorus compounds, which essentially comprise phosphomonoesters, inositol phosphates, phospholipids and nucleic acids, is only usable for plant growth after being converted into inorganic phosphates.

Furthermore, there are inorganic phosphorus compounds which are physically bound to a fibrous matrix, such as cellulose. These are likewise only liberated by biological decomposition processes in the soil where the fibrous matrix is decomposed and then are usable for plants. The biological decomposition processes in the soil are neither predictable nor controllable, since they depend on specific local circumstances of the soil such as pH value, moisture, temperature, precipitation and activity of microorganisms, and similar parameters.

The solubility of the inorganic phosphorus compounds in organic residues is chiefly dependent on their binding to divalent ions, such as calcium ions or magnesium ions. Thus, for example, in residues which have a high calcium concentration, such as swine manure, chicken manure or fermenting residue, the formation of calcium phosphate is thermodynamically preferred over other phosphates, depending on a pH value and a composition of the solution. Calcium phosphate is present as a solid in the residues and consequently it can be separated from the solid phase.

It is known how to use phosphatase-containing enzymes for the analytical determination of organic phosphorus compounds in manure and in the soil (see He et al. J. Environ. Qual., 2001.30: page 1685-1692 and Turner et al., Soil Biology and Biochemisty, 2002. 34(1): pages 27-35). However, the methods described in the aforementioned text passages are only useful for analytical purposes.

U.S. Pat. No. 6,776,816 B1 and WO 2006/081825 A1 describe methods which accelerate the conversion of organic phosphorus to inorganic phosphates. Furthermore, it is described how magnesium ammonium phosphates can be separated from animal dung by the addition of magnesium salts and selected enzymes, mentioning urease, uricase, allantoinase and phosphatase.

Drawbacks of these methods are that the enzymes are added as a liquid solution to the dung and therefore cannot be recovered or recycled. Consequently, the methods are very costly. Furthermore, the adding of magnesium salts means that the salt content of the dung increases. A further drawback of the known methods is that no solid/liquid separation takes place. Therefore, the magnesium ammonium phosphate salts remain in the dung and phosphorus is only separated from the liquid phase. The recovery of solid phosphate salts as a commercial end product is not described.

U.S. Pat. No. 5,993,503 A describes a method for dephosphatization of swine dung. For this, the swine dung is stored for the duration of at least one month at a temperature of 0 to 15° C. or moved continually for the duration of at least one week at 15° C. The precipitation of phosphates is prevented, since the pH value of the dung is adjusted to 8 and complexing agents are added to bind divalent ions. Furthermore, the document describes the breakdown of phytic acid in the dung by adding of enzymes, including ureases and phosphatases, in order to liberate the phosphates bound in the phytic acid so that they are present dissolved in the liquid.

Next, the dung is separated into a solid fraction and a liquid fraction. Phosphate is separated from the liquid fraction in the form of struvite (magnesium ammonium phosphate). The liquid fraction is concentrated down by means which require a large energy input, such as membrane methods, electrodialysis, or evaporation. Further drawbacks of the known method are: a long storage time at low temperatures, requiring a cooling of the dung and therefore entailing a high energy requirement. The enzymes are added to the dung as a liquid and therefore cannot be recovered or recycled. The adding of magnesium salts increases the salt content of the dung.

In U.S. Pat. No. 3,705,084 A it is described how enzymes, such as alkaline phosphatases, can be immobilized, that is, spatially fixed in gel particles, capsules, or also bounded reaction spaces.

WO 94/22770 A1 describes a method for removal of heavy metals, such as actinoids, in the form of insoluble phosphates. For this, a bioreactor is used, which contains immobilized phosphatase-producing microorganisms. The document describes how bacterial cultures can be used as phosphate donors for heavy metal enrichment. However, the resulting phosphates cannot be used as fertilizer, due to the heavy metal content.

Moreover, DE 10 2005 030 896 A1 reveals a centrifuge for the separation of a solid fraction and a liquid fraction of a dispersion, which contains biological material.

EP 0 265 027 A2 shows a method for the processing of liquid manure into a solid fraction on the one hand and a liquid fraction on the other hand, where the liquid manure is subjected to an anaerobic cleaning.

EP 1 829 829 A2 shows a device for the separation of biomass into a solid fraction and a liquid phase to be fermented for production of biogas.

U.S. Pat. No. 4,213,857 A shows an anaerobic fermentation process for rapid treatment of organic wastes, especially those which contain a lot of solids.

U.S. Pat. No. 4,765,900 A shows a method for the accelerated treatment of organic waste comprising liquid and solid fractions.

U.S. Pat. No. 6,776,816 B1 shows a method for the production of magnesium-ammonium-phosphate, which is suitable as a long-term fertilizer, and which is made for example by mixing of liquid manure with a predetermined amount of a magnesium-containing compound.

Against this background, the problem to be solved by the present invention is to provide a method which enables the dissolving of nutrients from organic residues in a largely continuous process in the form of fertilizer salts. Moreover, a solid fraction is to be dried at the end of an enzymatic process and be pelletized together with the obtained fertilizer salts, so that the method according to the invention produces a solid product, which can be used commercially as inorganic fertilizer and soil improver.

This problem is solved by the method according to the invention in that organic residues, such as stable manure or fermenting residues from anaerobic fermentation, are first subjected to a mechanical solid/liquid separation. In this way, advantageously a portion of the divalent ions contained in a first liquid phase are separated from a first solid phase.

The first solid phase so obtained is diluted with process water, so that a solution is formed with a dry substance content of preferably 5% and especially preferably 1%. If a concentration of divalent ions such as calcium or magnesium in the solution is still too high for a further use of the solution, the solution can be further subjected to the mechanical solid/liquid separation and again diluted with process water. In this way, the organic residues can be washed several times until the concentration of divalent ions in the solution is reduced so much that the further steps of the method can be carried out.

The solution still contains divalent ions, which are converted in a third step into hard to dissolve chemical compounds. A complete separation or inhibition of the divalent magnesium or calcium ions is required, since these have negative influence on the solubility of phosphorus required for the following steps of the method due to formation of hard to dissolve phosphate compounds.

The solution so pretreated is enzymatically digested in a fourth step, so that organically bound phosphorus is converted into inorganic compounds, preferably easily soluble phosphates. Enzymes are used for this, preferably phosphatases.

A following fifth step comprising a solid-liquid separation accomplishes a separation of the solution into a second solid phase and a second liquid phase, while the nutrients comprising phosphorus, nitrogen, calcium and magnesium are contained essentially in the liquid phase.

There follow further deposition processes in which the nutrients are successively separated from each other in the form of salts from the second liquid phase. The liquid phase so purified is returned as process water to the second step and thus used for the diluting, or the washing of the first solid phase.

The second solid phase is dried and pelletized. The nutrients recovered in the deposition processes are sent on to a pelletizing process. Thus, the method of the invention serves to produce an economically usable product in the form of a solid, organic fertilizer, whose nutrient composition and nutrient quantity can be adjusted to meet the requirements.

It is especially helpful to recover the process water from the second liquid phase. In this way, the water needed for the diluting and/or washing of the solid organic residues is obtained from the process itself, so that valuable resources are spared.

It is advantageous to accomplish the converting of the divalent ions into a hard to dissolve chemical compound by the adding of carbonates (salts of carbonic acid). By the adding of sodium bicarbonate, for example, the divalent ions are precipitated as magnesium carbonate or calcium carbonate. These carbonate compounds are hard to dissolve and the divalent ions cannot enter into a chemical bond with the dissolved phosphate.

An advantageous alternative for binding the divalent ions is to add complexing agents. The complexing agents comprise preferably humic acid, citric acid, nitrilotriacetic acid, alanine diacetic acid, citrates, gluconates and methylglycine diacetic acid. These substances are suitable to being attached to the divalent ions so that their reactivity is inhibited or reduced so much that they do not form a bond with dissolved phosphate.

Furthermore, it is advantageous for the enzymatic conversion to occur in a continuous-flow reactor. The continuous enzymatic mineralization of the phosphorus has the advantage over the prior art that the conversion takes place in relatively short time, at least 6 hours. In this way, large containers are avoided, in which the solution has to be treated for a long time, that is, stirred, heated or cooled. Therefore, the method of the invention leads to a reduction in energy demand and costs.

One advantageous embodiment calls for the reactor to have a substrate material, beads, carriers and/or a fill through which the solution flows, and enzymes are immobilized on the substrate material, the beads, the carriers, and/or the fill. Hereinafter, we shall only talk of the substrate material, but this term can also mean beads, carriers and/or fills. The advantage is that the immobilized enzymes are bound firmly to the substrate material or the fill and cannot pass over into solution. In this way, it is possible to recycle the enzymes. The enzymes remain bound to the fill for at least three months. After this, new enzymes can be immobilized on the substrate material. In this way, the substrate material can also be recharged with enzymes up to a hundred times, so that the costs incurred for the substrate material are recovered after less than three years.

Another advantageous embodiment calls for the reactor to be designed as a biocatalytic membrane reactor, where the enzymes are immobilized on membrane fibers. The use of a membrane reactor has the advantage that the conversion of the organic bound phosphorus into inorganically bound phosphorus and the solid-liquid separation occurs in a single step. In this way, costs for apparatus and fittings are saved in particular.

It is also advantageous for the immobilized enzymes to comprise phosphatases. Phosphatases are a group of enzymes which split the phosphorus compounds contained in the organic residues.

In addition, it is proposed to employ free and/or immobilized enzymes in the reactor, which are suitable for decomposing organic material. It is preferable for the immobilized or free enzymes to include cellulase, xylanase and/or glucanase. These enzymes are suitable for decomposing organic skeleton structures, so that the phosphorus enclosed in organic structures such as cells can be liberated.

One technically simple solution is to separate the inorganic phosphates as magnesium ammonium phosphate or as potassium magnesium phosphate. These phosphate compounds can be deposited from the second liquid phase by a method known from DE 10 2010 050 691 B3 in a reactor known from DE 10 2010 050 692 B3, without having to add from outside the process the ions required for this. In this way, an excessive salting of the second liquid phase is avoided.

Further features, application possibilities and benefits of the invention will emerge from the following description of sample embodiments of the invention, which are presented in the drawing. All features described or represented in themselves or in any given combination form the subject matter of the invention, regardless of their summarization in the patent claims or their back reference and also regardless of their phrasing or presentation in the specification and the drawing.

There are shown:

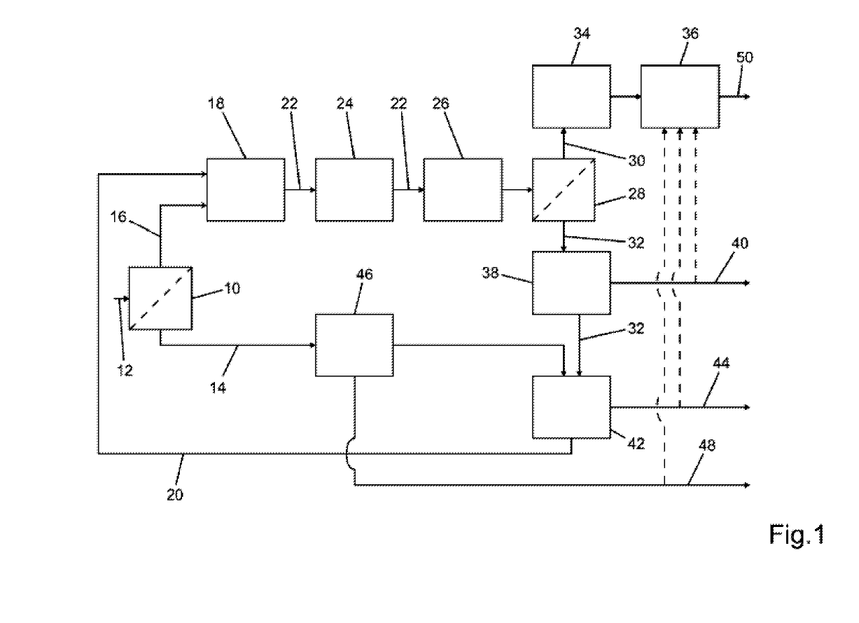

FIG. 1: a flow chart of the method of the invention in a first embodiment, and

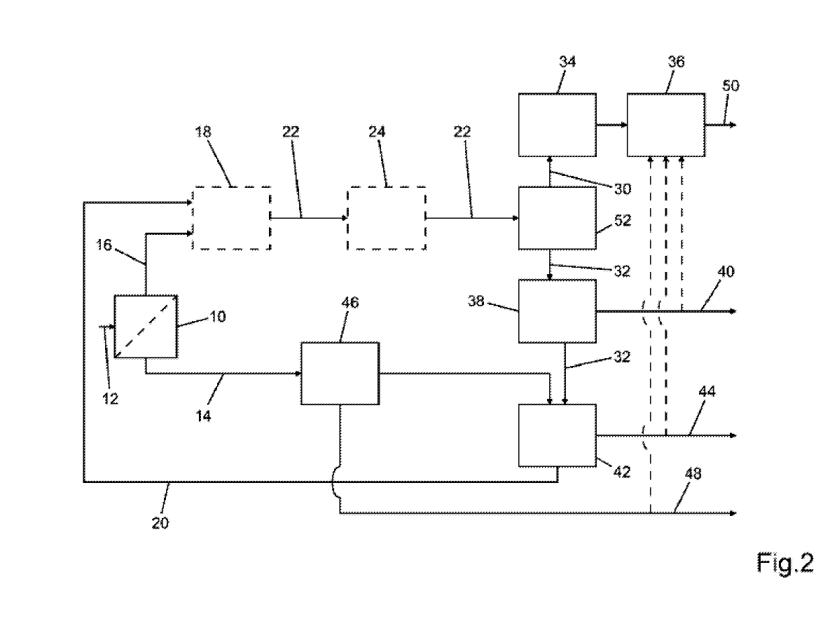

FIG. 2: a flow chart of the method of the invention in a second embodiment;

The same reference numbers are used for functionally equivalent elements and quantities in all the figures, even for different embodiments.

FIG. 1 shows the schematic flow chart of the method of the invention. In a first step 10, organic residues 12 are subjected to a mechanical separating process. This separates a first liquid phase 14 from a first solid phase 16. The first liquid phase 14 has a low phosphorus concentration and a high concentration of other ions, such as calcium or magnesium. A separate of divalent ions, including calcium and magnesium, is beneficial, sine these negatively affect the later steps.

The first solid phase 16 is mixed in a second step 18 with process water 20. The process water 20 is recycled from steps to be explained later on. The result is a solution 22 having a dry substance content of 5% or less, preferably 1%.

It is conceivable to return the solution 22 once more to the first step 10, if a concentration of a component of the solution 22 which is separated in step 10 with the first liquid phase 14 is too high.

After this, the solution 22 is taken to a third step 24. In this third step 24, divalent ions contained in the solution 22 are converted into a hard to dissolve compound.

It is preferably for the third step 24 to involve the adding of salts of carbonic acid, such as sodium bicarbonate, under stirring, to the solution 22 contained in a receptacle. In this way, the divalent ions are preferably separated as carbonates. The quantity of bicarbonate which is added depends essentially on the concentration of the divalent ions and the concentration of the salts of carbonic acid in the solution 22.

Alternatively, it is also conceivable to add complexing agents to the solution 22 in the third step 24, such as humic acid or citric acid.

Both alternatives, the adding of salts of carbonic acid or the adding of complexing agents, result in divalent ions such as calcium ions or magnesium ions becoming bound and thus having no negative impact on the solubility of phosphorus. Therefore, both alternatives bring about a raising of the phosphorus concentration in the solution 22.

A fourth step 26 involves an enzymatic treatment of the solution 22. In the fourth step 26, the organic phosphorus compounds are converted into inorganic phosphates. The fourth step 26 takes place in a continuous-flow reactor. The reactor has a substrate material, such as synthetic resin beads. Enzymes, preferably phosphatases, are immobilized in known manner on this substrate material. The solution 22 is mixed in the reactor with the enzymes immobilized on the fill. The enzymes help to convert the organic phosphorus compounds in the solution 22 into inorganic phosphorus compounds. Due to their solubility, the inorganic phosphorus compounds pass over into a second liquid phase of the solution 22.

A reaction time for the fourth step 26 amounts to at least six hours. After six hours, the mineralization of the phosphorus is complete. A process temperature amounts to preferably 20 to 50° C and a pH value of 5 to 10 is preferred.

It is also conceivable to employ enzymes which break down organic substances, such as cellulases or xylanases. In this way, phosphorus enclosed in the organic substances is liberated.

After the mineralization of the phosphorus occurring in the fourth step 26, a second solid-liquid separation occurs in a fifth step 28. With a mechanical separation process, the solution 22 which has left the reactor of the fourth step 26 is separated into a second solid phase 30 and a second liquid phase 32.

The second solid phase 30 is dried, preferably thermally, in a sixth step 34 and then pelletized in a seventh step 36.

The second liquid phase 32 has a substantially larger phosphate concentration than a liquid phase of the solution 22 not yet having gone through the third step 24, the separation of the divalent ions, and the fourth step 25, the enzymatic mineralization of the phosphorus.

The second liquid phase 32 is the starting solution for a first deposition process 38. Due to the high phosphorus concentration in the second liquid phase 32, no previous concentrating of the second liquid phase 32 is required for the first deposition process 38. The phosphorus is deposited from the second liquid phase 32 in the form of phosphates 40, including struvite or magnesium ammonium phosphate, MAP), K-struvite (potassium magnesium phosphate, KMP) or calcium phosphate.

One preferred method for deposition of the aforementioned phosphates 40 is an electrochemical method as is described in DE 10 2010 050 691 B3. The preferred method takes place preferably in a reactor known from DE 10 2010 050 692 B3.

This method requires no additional adding of magnesium salts or bases such as sodium hydroxide. All ions required for the deposition of the phosphates 40 are produced in the reactor itself. This prevents a salting of the second liquid phase 32.

For organic residues 12 which have a high ammonium concentration, such as fermenting residues, the second liquid phase 32 after the first deposition process 38 still contains substantial amounts of ammonium. In this case, the second liquid phase 32 is subjected to a second deposition process 42. In this, the ammonium contained in the second liquid phase 32 is precipitated in the form of ammonium salts 44, such as ammonium sulfate.

The second liquid phase 32 after this second deposition process 42 is practically free of nutrients and is returned as process water 20 to the second step 18.

The first liquid phase 14 separated in the first step 10 contains but little phosphorus, yet high concentrations of other ions such as calcium or magnesium. In a third deposition process 46, these other ions are deposited in the form of salts 48 from the first liquid phase 14.

After the third deposition process 46, the first liquid phase 14 is taken to the second deposition process 42, as described above, and the ammonium still contained in the first liquid phase 14 is precipitated. The first liquid phase 14 so purified is returned as process water 20 back to the second step 18.

The phosphate salts 40, ammonium salts 44 and calcium and/or magnesium salts 48 obtained in the deposition processes 38, 42 and 46 are added in the seventh step 36 to the second solid phase 30 being pelletized. This results in a product 50 which constitutes an organic fertilizer whose nutrient content can be adjusted by the adding of phosphate salts 40, ammonium salts 44 and calcium and magnesium salts 48 in desired manner to meet the requirements.

A second embodiment of the method of the invention is shown schematically in FIG. 2. The second embodiment differs from the first embodiment described above and presented in FIG. 1 in that the fourth step 26, involving the enzymatic mineralization of the phosphorus, and the fifth step 28, involving the solid-liquid separation, are combined into a single step 52.

Step 52 takes place preferably in a membrane reactor. The phosphates-containing enzymes are immobilized here on membrane fibers. The solution flows through the membrane, coming into contact with the enzymes, which bring about a mineralization of the phosphate. A liquid fraction of the solution 22 with the nutrients dissolved therein, such as phosphorus, ammonia, calcium and magnesium, passes through the membrane and is thus separated as a second liquid phase 32 from the second solid phase 30, which is held back by the membrane.

The reaction time for the single step 52 is at least 6 hours. The process temperature is preferably 30 to 50° C. and a preferred pH value is between 5 and 9.

It is conceivable in the single step 52 to immobilize enzymes which are suitable for breaking down organic substances, such as cellulase orxylanase, to the membrane fibers.

A preparation of the solution 22 before the single step 52 and the further processing of the second solid phase 30 and the second liquid phase 32 after the single step 52 is done as explained for the first embodiment presented in FIG. 1.

1. Method for recovery of organic and inorganic phosphorus compounds from solid components of organic residues (12) comprising the following steps:
separation of the organic residues (12) into a first solid phase (16) and a first liquid phase (14), by means of a mechanical separation process (10);
mixing of the first solid phase (16) with a process water (20) to form a solution (22);
conversion of divalent ions contained in the solution (22) into hard to dissolve chemical compounds;
enzymatic conversion of organic phosphorus compounds into inorganic phosphates in the solution (22);
separation of the solution (22) into a second solid phase (30) and a second liquid phase (32);
separation of inorganic phosphates (40) from the second liquid phase (32);
separation of ammonium salts (44) from the second liquid phase (32);
separation of the divalent ions contained in the first liquid phase (14) in the form of salts (48);
drying of the second solid phase (30); and
mixing and pelletizing of the second solid phase (30) with phosphate salts (40), ammonium salts (44) and salts (48) of the divalent ions that were recovered in preceding deposition processes (38, 42, 46).

2. Method according to claim 1, characterized in that the process water (20) is obtained from the second liquid phase (32).

3. Method according to claim 1, characterized in that the converting of the divalent ions into a hard to dissolve chemical compound is done by adding of carbonates.

4. Method according to claim 1, characterized in that the converting of the divalent ions into a hard to dissolve chemical compound is done by adding of complexing agents.

5. Method according to claim 4, characterized in that the complexing agents include humic acid, citric acid, nitrilotriacetic acid, alanine diacetic acid, citrates, gluconates and/or methylglycine diacetic acid.

6. Method according to claim 1, characterized in that the enzymatic conversion occurs in a continuous-flow reactor.

7. Method according to claim 6, characterized in that the continuous-flow reactor has a fill through which the solution (22) flows and enzymes are immobilized on the fill.

8. Method according to claim 6, characterized in that the continuous-flow reactor is designed as a biocatalytic membrane reactor, and the enzymes are immobilized on membrane fibers.

9. Method according to claim 8, characterized in that immobilized enzymes include phosphatases.

10. Method according to claim 8, characterized in that immobilized enzymes are suitable to breaking down organic matter.

11. Method according to claim 1, characterized in that the phosphate salts (40) are inorganic and are recovered as magnesium ammonium phosphate (MAP), or as potassium magnesium phosphate (KMP), or calcium phosphate.

12. Method according to claim 2, characterized in that the converting of the divalent ions into a hard to dissolve chemical compound is done by adding of carbonates.

13. Method according to claim 2, characterized in that the converting of the divalent ions into a hard to dissolve chemical compound is done by adding of complexing agents.

14. Method according to claim 13, characterized in that the complexing agents include humic acid, citric acid, nitriliotriacetic acid, alanine dicetic acid, citrates, gluconates and/or methylglycine diacetic acid.

15. Method according to claim 2, characterized in that the enzymatic conversion occurs in a continuous-flow reactor.

16. Method according to claim 15, characterized in that the continuous-flow reactor has a fill through which the solution (22) flows and enzymes are immobilized on the fill.

17. Method according to claim 15, characterized in that the continuous-flow reactor is designed as a biocatalytic membrane reactor, and the enzymes are immobilized on membrane fibers.

18. Method according to claim 17, characterized in that immobilized enzymes include phosphatases.

19. Method according to claim 17, characterized in that immobilized enzymes are suitable to breaking down organic matter.

20. Method according to claim 2, characterized in that the Phosphate salts (40) are inorganic and recovered as magnesium ammonium phosphate (MAP), or as potassium magnesium phosphate (KMP) or calcium phosphate.
